(19) United States
(12) Patent Application Publication
(10) Pub. No.: US 2007/0281072 A1
(43) Pub. Date: Dec. 6, 2007

(54) Coating a workpiece using a metering device and workpieces coated with this metering device

(75) Inventors: Timothy O'Connor, Claregalway (IE); Gabriel Sobrino, Kinvara (IE); Liam Ward, Ballinasloe (IE)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/447,175

(22) Filed: Jun. 6, 2006

(51) Int. Cl. A61L 33/00; B05D 3/12

(52) U.S. Cl. 427/2.1; 427/2.24; 427/356; 427/359

(57) ABSTRACT

The present invention is directed to methods, processes, and systems for selectively coating portions of a workpiece as well as to workpieces that have themselves been coated in accord with the invention. Under methods and processes of the invention, a target surface of a workpiece may be positioned in contact with a roller to coat a target surface of the workpiece. In some embodiments, the roller may also be positioned in contact with a metering device during portions or all of the treating and coating process. The workpiece may be an implantable medical device and the coating may include therapeutic, the workpiece may be other devices as well.

### TECHNICAL FIELD

The present invention generally relates to coated workpieces and methods and systems for selectively coating a workpiece with a metering device. More specifically, the present invention relates to workpieces, such as implantable medical devices, and methods and systems for coating these medical devices, wherein a treatment or other coating is selectively applied to some or all surfaces of the workpiece during a coating process.

### BACKGROUND

Coating workpieces is an often repeated procedure in contemporary manufacturing. Workpieces may be coated by methods that include tumble coating, spray coating, dip coating, and electrostatic spraying. During each of these procedures coating is applied to the workpiece prior to the workpiece being used for an intended purpose.

When the workpiece is formed partially or completely out of lattice struts or some other open framework, each of the faces of these struts or framework is exposed to the coating and coated during the coating methods listed above. By exposing each face of the workpiece to the coating being applied, each exposed face will be covered during the coating process.

When the workpiece being coated is an implantable medical device, such as a stent, all faces of the struts that comprise the stent are coated when using the coating systems identified above. For example, when dip coating is used, each face of the stent struts will be exposed to the coating. This coating will remain when the stent is removed from the dip and will dry on each face of the struts. Coating may also remain in the spaces between the struts. This phenomenon is sometimes called “webbing.” Here, not only are the individual struts covered, but some or all of the spaces between the struts are spanned by the coating as well.

### BRIEF DESCRIPTION

The present invention is directed to methods, processes, and systems for coating portions of a workpiece as well as to workpieces that have themselves been coated in accord with the invention. In accord with the invention, for example, some or all outer surfaces of a workpiece, such as a medical implant, may be coated with a therapeutic while inner surfaces of the implant, which are not targeted for coating, may not be coated.

Under methods and processes of the invention, a workpiece may be positioned in contact with a roller to coat a target surface of the workpiece. During portions of or all of the treating and coating process, the roller may also be positioned in contact with a metering device. This metering device may define a series of apertures or other openings that allow a portion of the coating which contacts the metering device to pass through the metering device. Moreover, the portion of the coating that does not initially pass through the metering device may remain behind until it may too pass through the apertures. In accordance with embodiments of the present invention, any one of the workpiece, the roller, and/or the metering device may be rotated. As noted, the workpiece may be an implantable medical device and the coating may include therapeutic, the workpiece may be other devices as well.

The invention may be embodied in numerous devices and through numerous methods and systems. The following detailed description, which, when taken in conjunction with the annexed drawings, discloses examples of the invention. Other embodiments, which incorporate some or all of the features as taught herein, are also possible.

### BRIEF DESCRIPTION OF THE DRAWINGS

Referring to the drawings, which form a part of this disclosure:

FIG. 1a shows a metering device that may be employed in accord with the present invention;

FIG. 1b shows another metering device that may be employed in accord with the present invention;

FIG. 1c shows another metering device that may be employed in accord with the present invention;

FIG. 1d shows another metering device that may be employed in accord with the present invention;

FIG. 2a is a cross-sectional view of a portion of a coated strut from a medical device that has been coated in accord with the present invention;

FIG. 2b is a cross-sectional view showing the coated strut of FIG. 2a after a second coating has been applied as may be employed in accord with the present invention;

FIG. 2c is a side-view of an arterial stent, which is a medical device that may be coated in accord with the present invention;

FIG. 3a shows a meyer bar and a rotating member that may be employed in accord with the present invention;

FIG. 3b is an enlarged view of the meyer bar of FIG. 3a during the coating process in accord with the present invention;

FIG. 4a shows a system of coating a workpiece with a roller and a metering device that may be employed in accord with the present invention;

FIG. 4b shows another system of coating a workpiece with a roller, a metering device, and a doctor blade that may be employed in accord with the present invention;

FIG. 4c shows still another system of coating a workpiece with a roller and a metering device that may be employed in accord with the present invention; and

FIG. 5 shows a flow chart illustrating method steps that may be employed with embodiments of the present invention.

### DETAILED DESCRIPTION

The present invention regards coating one or more surfaces of a workpiece while not coating other surfaces of the workpiece. In some embodiments this may include coating the outside or side surfaces of the workpiece while not coating the inside surfaces of the workpiece. By coating in this fashion the amount of coating resident on the workpiece may be reduced. For example, if the workpiece is a medical implant and the coating contains therapeutic a reduction in coating may allow the therapeutic to be delivered in a more targeted fashion after the stent is implanted at a target site. The limited use of coating can also conserve coating materials, which themselves may be valuable.

A desired uniform thickness of coating may be applied to a target surface of a workpiece using a roller and a metering device in accord with the present invention. For example, the metering device may contact a roller to regulate the thickness of the coating. Each of the workpiece, the roller, and the metering device may be rotated to facilitate the coating of one or more surfaces of the workpiece. The roller may be rotated within a coating reservoir or otherwise positioned in communication with a coating source. A plurality or combination of metering devices may also be used. For example, a meyer rod may be used in combination with a smooth rod.

The meyer rod is a rod with wire wrapped around an outside surface. The meyer rod is generally used to regulate coating solution by controlling the weight and/or thickness of the coating. The meyer rod may improve the accuracy of achieving predetermined coating thicknesses and may also improve the uniformity of resulting coating thicknesses. The meyer bar rod and wire may be any suitable size. For example, the wire can be between about 2 and 150 mils. There are spaces or interstices formed between adjacent wire turns. The thickness of these spaces is determined by the thickness or diameter of the wire. The thickness of the wire, in turn, determines the thickness of the coating transferred from the roller to the target surface of the workpiece.

The rods and wires discussed herein may be any suitable metallic and/or polymeric material such as stainless steel. The outer surface of the rods and wires may also be coated with a non-stick material. For instance, PTFE and Teflon™ may be used. Further, the outer surface of the rods and wires may also be plated. For example, the rods and wires may be plated with chrome, nickel, or titanium nitride. Plating the rod and wire may facilitate coating by providing a relatively smooth contact surface. Further, plating the rod and wire may also reduce wear, by reducing abrasion, and increase the life span of the metering device.

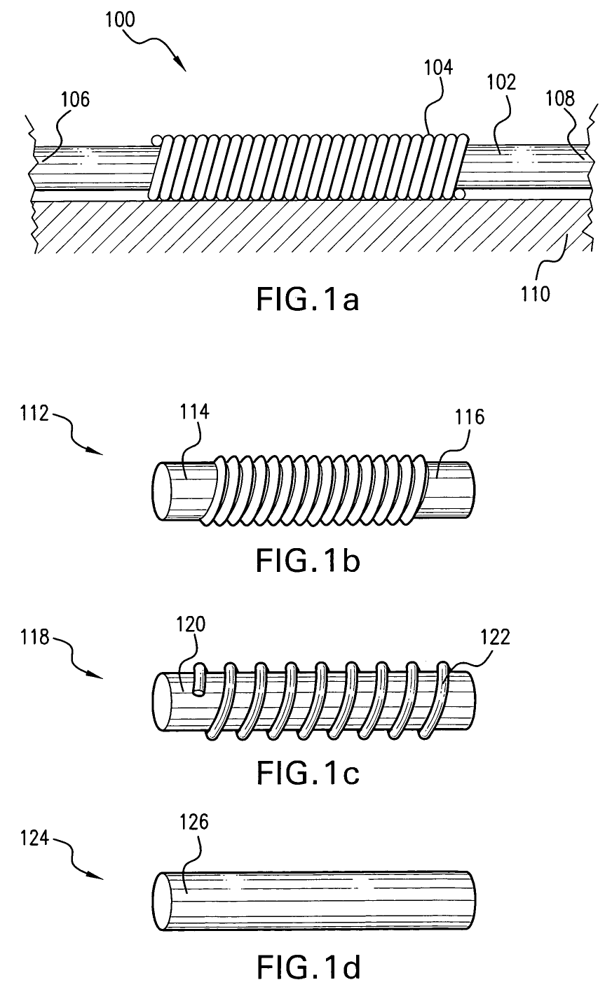

FIG. 1a shows a metering device 100 that may be employed in accord with the present invention. Evident in FIG. 1a are a rod 102 including a wire 104 that may be closely wound around the circumference from one end 106 of the rod 102 to the other end 108. Also shown in FIG. 1a is a portion of a roller 110 that may be used for coating a target surface of a workpiece. The roller 110 may have coating, such as a therapeutic, on a surface thereof. The rod 102 and wire 104 may be used to regulate coating thickness. In other words, the rod 102 and wire 104 may remove or control an amount of coating on the roller 110. Consequently, after moving to a position beyond the metering device 100, the roller 110 may transfer the coating to a target surface of a workpiece.

FIG. 1b shows another metering device 112 that may be employed in accord with the present invention. In FIG. 1b, the metering device 112 may be a rod 114 including threads 116 integrally formed on a surface thereof. The threaded rod 114 may be used in applications where, for example, greater coating thicknesses and higher viscosity coatings are used. Alternatively, as shown in FIG. 1c, another metering device 118 may be used having a rod 120 with wires 122 that may be wound so that relatively larger spaces, comparison to FIG. 1a, exist between adjacent wires 122. The metering device 118 of FIG. 1c may also be used, for instance, in applications where greater coating thicknesses and higher viscosities are used.

FIG. 1d shows another metering device 124 that may be employed in accord with the present invention. Evident in FIG. 1d, the rod 126 may have a substantially smooth surface. The smooth surface rod may be used, for example, in conjunction with one of the metering devices 100, 112, 118 discussed herein to facilitate coating uniformity.

In other embodiments, which are not shown, a plurality of additional wires may be wrapped around the rod of the metering device in a variety of other ways. For example, a second wire may be wound around the rod. Further, the second wire may be of a smaller diameter and wrapped around the grooves of the first rod. The multiple wire wrapped rods may be used, for example, in applications that apply a relatively thick coating. Additionally, any combinations of the above-identified metering devices may be plausible.

In FIGS. 1a-1d, the rods 102, 114, 120, and 126 are substantially cylindrical, however, the rods 102, 114, 120, and 126 may be any suitable shape and size. In the examples of FIGS. 1a-1d, the rods 102, 114, 120, and 126 and wires 104, 122 may be constructed of stainless steel.

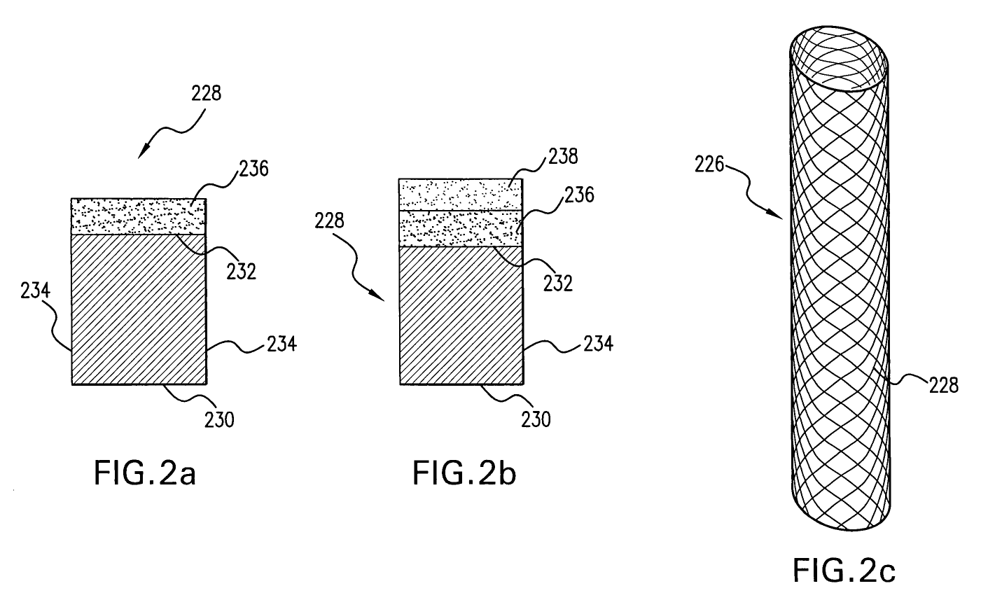

FIG. 2a is a side sectional view of a strut 228 of a stent 226 that may be coated in accord with the present invention. The strut 228 in FIG. 2a has an inner surface 230, an outer surface 232, and two cut faces 234. Also shown on the strut 228 is a coating 236. As can be seen, the coating 236, covers only one face of the strut 228.

FIG. 2b shows another example of how a coating may be applied in accord with the invention. In FIG. 2b, a first coating 236 and a second coating 238 have been applied to the strut 228. As can be seen, the first coating 236 is in contact with the strut 228 while the second coating 238 is in contact with the first coating 236 and further covers the outer surface 232 of the strut 228. This second coating 238 may be applied in accord with the processes and methods of the present invention. It may also be applied with different methods and processes. In this example, as well as with the others described herein, if a second coating 238 is employed this coating may comprise the same materials as the first coating 236 and it may differ from the materials used for the first coating 236. In still other examples the coating may be applied in other patterns as well. For example, it may be applied to opposing cut faces 234 and not the outer surface 232, likewise it may be applied to both cut faces 234 and the outer surface 232. In a exemplary embodiment, the outer surface 232 is coated and the two cut faces 234 as well as the inner surface 230 are not.

FIG. 2c is a side view of an implantable aortic stent 226 including a lattice portion that may be coated in accord with the invention. The stent 226 may be porous or have portions thereof that are porous. The struts 228 shown in FIGS. 2a and 2b are struts 228 that may comprise and make up this stent 226. While the workpiece shown in these initial figures is a stent 226, many other workpieces may be coated in accord with the invention. For example, other medical devices that may be coated include filters (e.g., vena cava filters), stent grafts, vascular grafts, intraluminal paving systems, implants and other devices used in connection with drug-loaded polymer coatings. Likewise, the workpeice may not be an implantable medical device but may, instead, be another piece that needs to be coated only on certain pre-selected surfaces. In some instances these medical devices or other workpieces may be made from conductive materials and in other instances they may not be. For example, they may be made from polymers or ceramics.

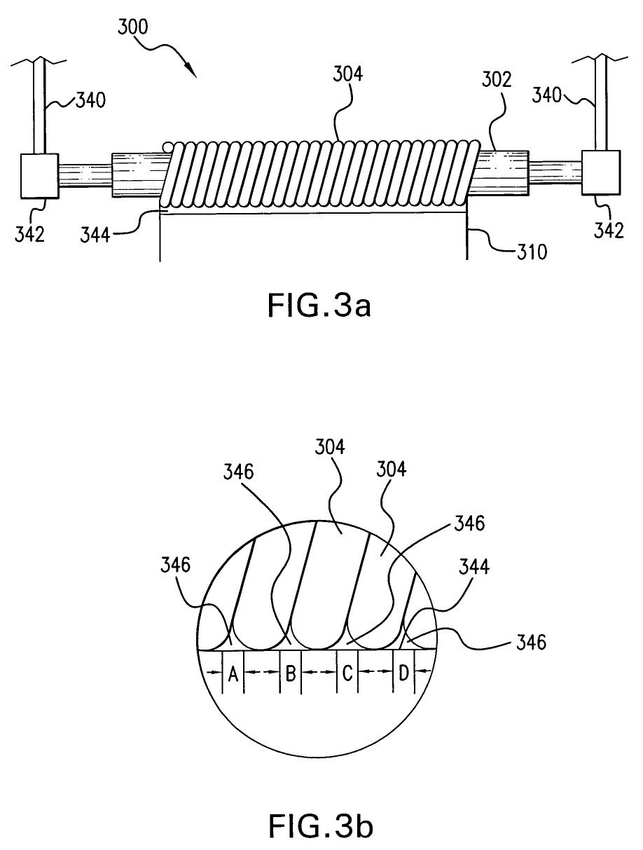

FIG. 3a shows a meyer bar 300 which may be employed with the embodiments of the present invention. In this example, the meyer bar 300 may be connected to supports 340 and also may be in contact with a portion of a roller 310. The meyer bar 300 may include a rod 302 with wire 304 wound around the circumference from one end of the rod to the other end. The rod 302 and wire 304 may be made of any suitable material such as stainless steel. For example, the rod 302 may be 3′ 1/16″ inch stainless steel. The meyer bar 300 may be rotated and the rod 302 of the meyer bar 300 may even be rotatably connected to a drive mechanism 342. The drive mechanism 342 can use an electrical, mechanical, or hydraulic drive source, and may use combinations thereof. For example, motors, endless belts, gearing, or hand cranks may be used.

The roller 310 may also be rotatable and may fluidly communicate with a coating source (not shown). The coating source may supply coating to the roller 310 to a coat portion of the roller. Then, the coated portion of the roller 310 may contact the meyer bar 300 prior to the roller 310 contacting a target surface of the workpiece. The meyer bar 300 may act as a squeegee to remove or control the amount of coating 344 located on a portion of the roller 310.

As illustrated in FIG. 3b, a plurality of spaces 346 may be formed between adjacent wires 304. The thickness of the wires 304 may determine the thickness of the spaces 346. These spaces 346 may have thicknesses A, B, C, and D which may control the thickness of the coating 344 transferred to the target surface of the workpiece. Therefore, in accord with the present invention, the thickness of the coating 344 may depend on the diameter or gauge of the wire 304, which may determine the thickness A, B, C, and D of the spaces 346. As a portion of the roller 310 travels past the meyer bar 300, the meyer bar 300 may squeegee off all but the amount of coating 344 located in the spaces 346.

As stated above, the wet thickness of the coating may be substantially controlled by selecting a desired thickness or gauge of the wire. For example, the wet thickness of the coating may be about 0.1 times the wire diameter. Numerous wire sizes may

| | |
|---|---|
| Wire Size | Wet Film |

| Mils (0.001″) | Millimeters | Mils (0.001″) | Microns |
|---|---|---|---|
| 2 | 0.05 | 0.18 | 4.47 |
| 3 | 0.08 | 0.27 | 6.88 |
| 4 | 0.1 | 0.36 | 9.14 |
| 5 | 0.13 | 0.45 | 11.43 |
| 6 | 0.15 | 0.54 | 13.72 |
| 7 | 0.18 | 0.63 | 16 |
| 8 | 0.2 | 0.72 | 18.79 |
| 9 | 0.23 | 0.81 | 20.57 |
| 10 | 0.25 | 0.9 | 22.64 |
| 12 | 0.3 | 1.08 | 27.43 |
| 14 | 0.36 | 1.26 | 32 |
| 16 | 0.41 | 1.44 | 36.68 |
| 18 | 0.46 | 1.62 | 41.15 |
| 20 | 0.51 | 1.8 | 45.72 |
| 22 | 0.56 | 1.95 | 50.29 |
| 24 | 0.61 | 2.16 | 54.5 |
| 26 | 0.66 | 2.34 | 59.44 |
| 28 | 0.71 | 2.52 | 64.01 |
| 30 | 0.76 | 2.76 | 69.14 |
| 32 | 0.81 | 2.88 | 73.15 |
| 34 | 0.86 | 3.06 | 77.72 |
| 36 | 0.91 | 3.24 | 82.3 |
| 38 | 0.97 | 3.42 | 88.87 |
| 40 | 1.02 | 3.66 | 91.44 |
| 42 | 1.07 | 3.78 | 96.01 |
| 44 | 1.12 | 3.96 | 100.58 |
| 46 | 1.17 | 4.14 | 105.16 |
| 48 | 1.22 | 4.32 | 109.73 |
| 50 | 1.27 | 4.5 | 114.3 |
| 55 | 1.4 | 4.95 | 125.73 |
| 60 | 1.5 | 5.46 | 137.16 |
| 65 | 1.65 | 5.85 | 145.59 |
| 70 | 1.78 | 6.3 | 160.02 |
| 75 | 1.91 | 6.75 | 171.45 |
| 80 | 2.03 | 7.2 | 182.88 |
| 85 | 2.16 | 7.65 | 194.31 |
| 90 | 2.29 | 8.1 | 205.74 |
| 95 | 2.41 | 8.55 | 217.17 |
| 100 | 2.54 | 9 | 228.8 |
| 105 | 2.67 | 9.45 | 240.03 |
| 110 | 2.79 | 9.9 | 251.46 |
| 115 | 2.92 | 10.35 | 262.39 |
| 120 | 3.05 | 10.8 | 274.32 |
| 125 | 3.18 | 11.25 | 285.75 |
| 130 | 3.3 | 11.7 | 297.18 |
| 135 | 3.43 | 12.15 | 308.61 |
| 140 | 3.56 | 12.6 | 320.04 |
| 145 | 3.64 | 13.05 | 331.47 |
| 150 | 3.81 | 13.5 | 342.9 |

be available to produce a desired wet film thickness. Wet thickness may be controlled within about 0.1 mils or 2.5 microns. For example, the following table provides exemplary wire sizes and wet film thicknesses in both U.S. standard and metric units of measure which may be suitable.

Comparatively, the dry thickness of the coating may be determined in part by the solids concentration and evaporation rate of the coating solution. The characteristics of the coating (e.g. viscosity) may also be relatively important in determining the dry thickness of the coating solution.

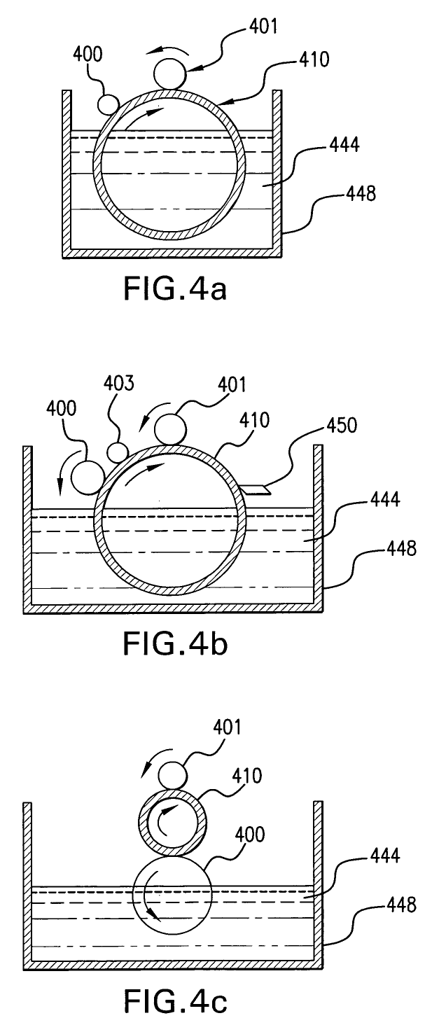

FIG. 4a shows a system for coating a target surface of a workpiece 401 using a roller 410 and a metering device 400 that may be employed in accord with the present invention. In this example, the metering device 400 may be stationary. The roller 410 may be at least partially positioned in a reservoir 448 having coating 444. Also evident in FIG. 4a, the roller 410 may be rotatable in the clockwise direction and the workpiece 401 may be rotatable in the counterclockwise direction. However, the roller 410 and the workpiece 400 can rotate in the same direction. Alternatively, the roller 410 may rotate in the counterclockwise direction and the workpiece 401 in the clockwise direction.

FIG. 4b shows another a system for coating a target surface of a workpiece 401 using a roller 410, a plurality of metering devices 400, 403, and a doctor blade 450 that may be employed in accord with the present invention. In this example, the roller 410 may be at least partially positioned within a reservoir 448 having coating 444. Additionally, in this instance, the roller 410 may rotate in the clockwise direction, one metering device 400 and the workpiece 401 may be rotated in the counterclockwise direction, and the other metering device 403 may be stationary. However, other suitable arrangements may be plausible.

FIG. 4c shows still another system of coating a target surface of a workpiece 401 using a roller 410 and a metering device 400 that may be employed in accord with the present invention. In this embodiment, the metering device 400 may be at least partially positioned within a reservoir 448 having coating 444. In this example, the metering device 400 and workpiece 401 may be rotated counterclockwise. The roller 410 may be rotated clockwise. In this embodiment, the roller 410 may be located outside of the reservoir 448 and may be coated by the metering device 400. However, other arrangements are possible.

Other suitable examples not shown in FIGS. 4a-4c may also be used. For example, any one of the roller 410, metering devices 400, 403, 450, and/or the workpiece 401 may be rotated in any desired direction or may also be not rotated at all.

Moreover, any combination of metering devices 400, 403, 450, not limited to those shown, may also be used.

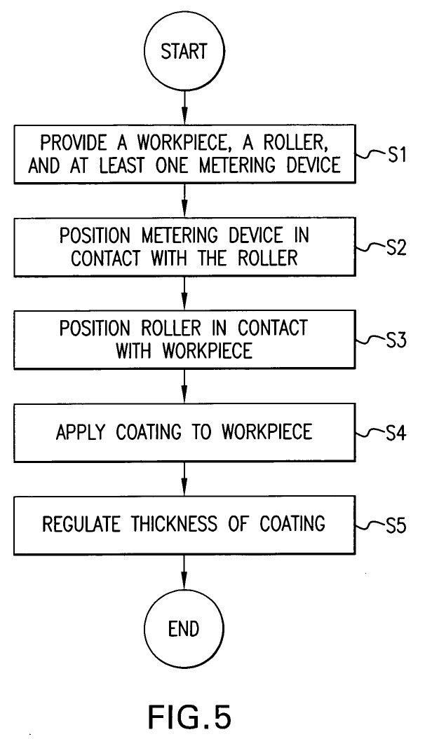

FIG. 5 shows a flow chart including method steps that may be employed with embodiments of the present invention to coat a target surface of a workpiece. In the example of FIG. 5a, step S1 may include providing a workpiece, a roller, and at least one metering device. Step S2 may include positioning a metering device in contact with a portion of the roller. S3 may include positioning a target surface of the workpiece in contact with a portion of the roller. S4 can include applying a therapeutic coating to a target surface of the workpiece via the roller. S5 may include controlling coating thicknesses on the workpiece using the metering device. In alternative embodiments, not shown, the sequence of steps may be reordered and steps may be added or removed. The steps may also be modified. Further, the steps may be repeated in continuous fashion.

While various embodiments have been described, other embodiments are plausible. It should be understood that the foregoing descriptions of various examples of the metering device and roller are not intended to be limiting, and any number of modifications, combinations, and alternatives of the examples may be employed to facilitate the effectiveness of the coating of target surfaces of the workpiece.

The coating, in accord with the embodiments of the present invention, may comprise a polymeric and or therapeutic agent formed, for example, by admixing a drug agent with a liquid polymer, in the absence of a solvent, to form a liquid polymer/drug agent mixture. A suitable list of drugs and/or polymer combinations is listed below. The term “therapeutic agent” as used herein includes one or more “therapeutic agents” or “drugs.” The terms “therapeutic agents” or “drugs” can be used interchangeably herein and include pharmaceutically active compounds, nucleic acids with and without carrier vectors such as lipids, compacting agents (such as histones), viruses (such as adenovirus, andenoassociated virus, retrovirus, lentivirus and α-virus), polymers, hyaluronic acid, proteins, cells and the like, with or without targeting sequences.

Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor ∀ and ∃, platelet-derived endothelial growth factor, platelet-derived growth factor, tumor necrosis factor ∀, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

As stated above, coatings used with the exemplary embodiments of the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

The polymer used in the exemplary embodiments of the present invention is preferably capable of absorbing a substantial amount of drug solution. When applied as a coating on a medical device in accordance with the present invention, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present invention to apply multiple layers of polymer coating onto a medical device. Such multiple layers are of the same or different polymer materials.

The polymer of the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers.

Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. A method of coating a workpiece, comprising:
providing a workpiece, a roller, and a metering device;
positioning a target surface of the workpiece in contact with the roller;
positioning the metering device in contact with the roller; and
applying a therapeutic coating having a thickness to the target surface of the workpiece via the roller,
wherein the metering device regulates the coating thickness and defines a plurality of apertures through which the coating passes prior to reaching the workpiece.

2. The method of claim 1, wherein the roller is positioned in a coating reservoir.

3. The method of claim 1, wherein the metering device is positioned in a coating reservoir.

4. The method of claim 1, wherein the roller is rotatable.

5. The method of claim 1, wherein the metering device is rotatable.

6. The method of claim 1, wherein the workpiece is rotatable.

7. The method of claim 1, wherein the metering device is a meyer bar including a rod and wire positioned around the rod.

8. The method of claim 7, wherein a diameter of the wire determines the thickness of the coating transferred to the workpiece.

9. A method of claim 1, further comprising providing a doctor blade in contact with the roller to remove coating.

10. The method of claim 1 wherein the target surface is an outer surface of the workpiece.

11. A method of coating a surface of a medical implant, the method comprising:
positioning a target surface of the medical implant in contact with a roller;
positioning a metering device in contact with the roller; and
applying a coating having a thickness to the medical implant via the roller,
wherein the metering device regulates the coating thickness and defines a plurality of apertures through which the coating passes prior to reaching the workpiece.

12. The method of claim 11, wherein the roller is positioned in a coating reservoir.

13. The method of claim 11, wherein the metering device is positioned in a coating reservoir.

14. The method of claim 11, wherein the metering device is a meyer bar including a rod and wire positioned around the rod.

15. The method of claim 14, wherein the diameter of the coil determines the thickness of the coating transferred to the workpiece.

16. A method of claim 11, further comprising providing a doctor blade in contact with the roller to remove coating.

17. A method of claim 11, wherein the coating is a therapeutic.

18. A method of coating a stent, the method comprising:
positioning a target surface of a stent in contact with a roller;
positioning a meyer bar in contact with the roller, the meyer bar including a rod and wire positioned around the rod; and
applying a coating having a thickness to the stent via the roller,
wherein the meyer bar regulates coating thickness. wherein the metering device regulates the coating thickness and defines a plurality of apertures through which the coating passes prior to reaching the workpiece.

19. The method of claim 18, wherein the roller is positioned in a coating reservoir.

20. The method of claim 18, wherein a diameter of the coil determines the thickness of the coating transferred to the workpiece.
